(19) United States
(12) Patent Application Publication
(10) Pub. No.: US 2014/0349245 A1
(43) Pub. Date: Nov. 27, 2014

(54) BITE TAB FOR DENTAL SENSORS

(71) Applicant: Gerald Gestetner, Montreal (CA)

(72) Inventor: Gerald Gestetner, Montreal (CA)

(21) Appl. No.: 14/287,131

(22) Filed: May 26, 2014

Related U.S. Application Data
(60) Provisional application No. 61/827,716, filed on May 27, 2013.

(51) Int. Cl. A61B 6/14

(52) U.S. Cl. CPC A61B 6/145
USPC 433/29

(57) ABSTRACT

A positioning bite tab for a dental sensor, said positioning bite tab comprising: a base element, said base element defining a substantially plate-shaped biting portion; and a strap movable between a strap open configuration and a strap closed configuration, wherein, in said strap open configuration, said strap is generally planar, and in said strap closed configuration, said strap forms a loop for receiving said dental sensor thereinto and securing said dental sensor thereto; said base element being attachable to said strap in a first orientation and in a second orientation, said biting portion being oriented differently relative to said strap in said first and second orientations.

### FIELD OF THE INVENTION

The present invention relates to the general field of radiography, and is more specifically concerned with a positioning bite tab for dental sensors, for example for a dental digital sensor.

### BACKGROUND

Digital x-ray sensors are used by dentists as a replacement to traditional X-ray methods. The sensor is covered by an infection control barrier and then placed in the patient's mouth in the desired area. The purpose of a bite tab is to have the sensor remain in the proper location throughout the X-ray process and not be moved around by the mouth. It is sometimes desirable to select the orientation and position of the sensor in the mouth. In order to reduce the number of parts that must be stocked by dentists, it would be advantageous to have a support for the sensor that would allow different positions and orientations of the sensor relative to the mouth. There is at least one such device commercialized, but this device is relatively complex, and therefore relatively expensive to manufacture. Furthermore, this device, by its design, is flexible, which can lead to undesired movements of the sensor in the mouth due to involuntary minor movements of the patient when the X-ray image is acquired. These movements can lead to blurry images.

Against this background, there exists a need in the industry to provide an improved positioning bite tab for dental sensors. An object of the present invention is therefore to provide such a positioning bite tab for dental sensors.

### SUMMARY OF THE INVENTION

In a broad aspect, the invention provides a positioning bite tab for a dental sensor, the positioning bite tab comprising: a base element, the base element defining a substantially plate-shaped biting portion; and a strap movable between a strap open configuration and a strap closed configuration, wherein, in the strap open configuration, the strap is generally planar, and in the strap closed configuration, the strap forms a loop for receiving the dental sensor thereinto and securing the dental sensor thereto; the base element being attachable to the strap in a first orientation and in a second orientation, the biting portion being oriented differently relative to the strap in the first and second orientations.

Advantageously, the proposed bite tab is relatively simple, relatively easy to manufacture and relatively inexpensive, while attaining the objective of allowing different positions and orientations of the sensor relative to the mouth of the patient.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawing:

FIG. 1, in an exploded perspective view, illustrates a positioning bite tab in accordance with an embodiment of the present invention, the bite tab including a base element and a strap, the strap being shown in a strap open configuration;

FIG. 2, in side midway cross-sectional view, illustrates the positioning bite tab shown in FIG. 1 with the base element and the strap thereof secured to each other in a first orientation, the strap being shown in a strap closed configuration;

FIG. 3, in side midway cross-sectional view, illustrates the positioning bite tab shown in FIGS. 1 and 2 with the base element and the strap thereof secured to each other in a second orientation, the strap being shown in the strap closed configuration.

### DETAILED DESCRIPTION

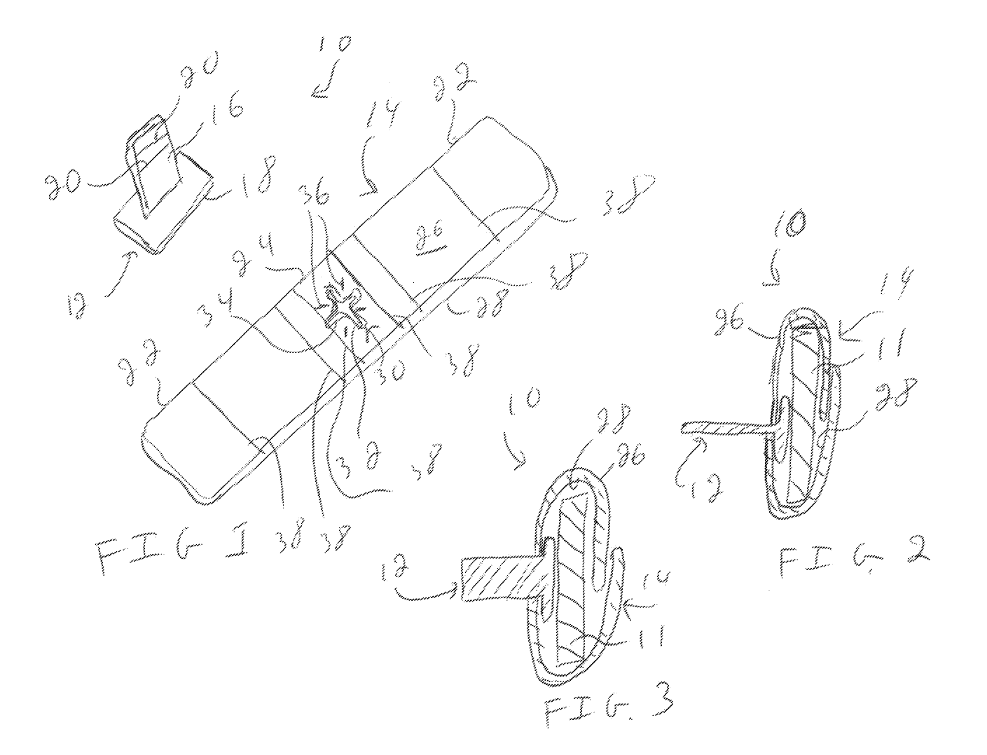

Referring to FIGS. 1 to 3, there is shown a positioning bite tab 10 for a dental sensor 11 (only seen in FIGS. 2 and 3). The positioning bite tab 10 includes a base element 12 and a strap 14. The base element 12 and the strap 14 are securable to each other. The base element 12 is relatively rigid, while the strap 14 is relatively flexible.

Referring more specifically to FIG. 1, the base element 12 defines a biting portion 16, which is typically substantially plate-shaped. The base element 12 also defines a base portion 18, which is also typically substantially plate-shaped. Typically, the biting and base portions 16 and 18 extend from each other generally perpendicularly relative to each other with the intersection of the biting and base portions 16 and 18 entirely spaced apart from the perimeter of the base portion 18.

In some embodiments of the invention, the biting and base portion 16 and 18 are integrally formed as a single piece of material, for example in plastic.

The biting portion 16 is configured and sized for being bitable between the teeth of a patient (both not shown in the drawings). In some embodiments of the invention, the biting portion 16 defines one or more grooves 20 extending thereinto and extending generally parallel to the base portion 18 for providing grip to the teeth.

The strap 14 is generally elongated and flexible. The strap 14 defines a pair of longitudinally opposed strap end sections 22 and a strap middle section 24 provided therebetween. The strap 14 also defines a strap first surface 26 and an opposed strap second surface 28. The strap 14 is movable between a strap open configuration (seen in FIG. 1) and a strap closed configuration (seen in FIGS. 2 and 3). In the strap open configuration, the strap 14 is generally planar. In the strap closed configuration, the strap 14 forms a loop for receiving the dental sensor 11 thereinto and securing the dental sensor 11 thereto, and the strap first surface 26 faces outwardly while the strap second surface 28 faces inwardly towards the dental sensor 11.

The strap middle section 24 defines a generally plus-shaped slot 30 extending therethrough between the strap first and second surfaces 26 and 28 for receiving the base element 12. More specifically, the slot 30 includes two rectilinear branches 32 and 34 that intersect at right angle relative to each other in their respective middles. Branch 34 is generally longitudinally oriented. In some embodiments of the invention, auxiliary slots 36 extend through the strap 14 between the strap first and second surfaces 26 and 28 obliquely between the branches 32 and 34, while remaining disjoint therefrom. The auxiliary slots 36 facilitates the deformation of the strap middle section 24 to allow insertion of the base element 12 in the slot 30 and a movement of the base element 12 in the slot 30 between two configurations, as detailed in further details hereinbelow.

The strap end sections 22 are provided with an adhesive of the strap second surface 28. Typically, the adhesive is strong enough to maintain the strap 14 in the strap closed configuration, while allowing opening of the loop formed by the strap 14 in the strap closed configuration. As seen for example in FIG. 2, in this strap closed configuration, the adhesive of one of the strap end sections 22 sticks to the dental sensor 11, while the adhesive of the other strap end section 22 sticks to the strap first surface 26. In some embodiments of the invention, the adhesive is covered by a removable flap (not shown in the drawings) before the strap 14 is used. In some embodiments of the invention, the surface of the base element 18 opposed to the biting element 16 is also covered with an adhesive for securing the sensor element 11 thereto.

In some embodiments of the invention, the strap 14 is provided with transversely oriented grooves 38 extending in the strap first surface 26. The grooves 38 provide fold lines to help in folding the strap 14 around the dental sensor 11 to obtain the strap closed configuration. These grooves 38 also serve as positioning marks to help properly position the dental sensor 11 along the strap 14 prior to folding.

In use, the base element 12 is first attached to the strap 14 with the strap 14 in the strap open configuration. The base element 12 is attachable to the strap 14 in a first orientation and in a second orientation. The biting portion 16 is oriented differently relative to the strap 14 in the first and second orientations. To that effect, the biting portion 16 is inserted in the slot 30, more specifically into one of the branches 32 and 34, from the strap second surface 28 towards the strap first surface 26 until the base portion 18 abuts against the strap second surface 28. The first and second orientations are selected depending on which ones of the branches 32 and 34 receives the biting portion 16. If desired, after attachment, the base element 12 can be rotated to achieve either one of the first and second orientations.

Subsequently, the strap 14 is folded around the dental sensor 11 into the strap closed configuration. The position of the dental sensor 11 relative to the base element 12 can be adjusted by moving the base element 12 along the dental sensor 11 and attaching the adhesive of the strap end section 22 that abuts against the dental sensor 11 at a suitable location therealong.

Although the present invention has been described hereinabove by way of preferred embodiments thereof, it can be modified, without departing from the spirit and nature of the subject invention as defined in the appended claims.

What is claimed is:

1. A positioning bite tab for a dental sensor, said positioning bite tab comprising:
a base element, said base element defining a substantially plate-shaped biting portion; and
a strap movable between a strap open configuration and a strap closed configuration, wherein, in said strap open configuration, said strap is generally planar, and in said strap closed configuration, said strap forms a loop for receiving said dental sensor thereinto and securing said dental sensor thereto;
said base element being securable to said strap in a first orientation and in a second orientation, said biting portion being oriented differently relative to said strap in said first and second orientations.
